(19) United States
(12) Patent Application Publication
(10) Pub. No.: US 2010/0105076 A1
(43) Pub. Date: Apr. 29, 2010

(54) ANALYSIS KIT COMPRISING AT LEAST TWO MOLECULARLY IMPRINTED POLYMERS AND AT LEAST ONE MARKER, AND METHOD OF ANALYSIS USING SAME

(75) Inventors: Celine Perollier, Rouen (FR); Kaynoush Naraghi, Rouen (FR); Sami Bayoudh, Rouen (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: Polyintell, Val de Rueil (FR)

(21) Appl. No.: 12/532,288

(22) PCT Filed: Mar. 20, 2008
(86) PCT No.: PCT/FR08/50487

§ 371 Date: Sep. 21, 2009

(30) Foreign Application Priority Data
Mar. 20, 2007 (FR) 0753941

(51) Int. Cl. G01N 33/573; G01N 21/75; G01N 33/00; G01N 33/92; G01N 33/02

(52) U.S. Cl. 435/7.4; 436/164; 436/111; 436/71; 436/95; 436/96; 436/86; 436/20

(57) ABSTRACT

The present invention relates to a kit for analysing at least one target molecule comprising at least a first molecularly imprinted polymer and a second molecularly imprinted polymer which are chemically identical or different, capable of interacting with the target molecule(s), and at least one marker, wherein said marker is either a competitive marker or marker capable of (i) of interacting with all or part of the recognition sites for the target molecule(s) of the second molecularly imprinted polymer and (ii) of being displaced from said recognition site(s) by the target molecule(s) when said molecularly imprinted polymer is placed in the presence of said target molecule(s), or an intrinsic marker or a constitutive unit of the second molecularly imprinted polymer, capable (i′) of interacting with all or part of the target molecule(s) when the latter interact(s) with all or part of its recognition sites for said target molecule(s) and (ii″) of consequently emitting a detectable signal.

The present invention relates to the area of molecularly imprinted polymers (MIPs), which can be used for the recognition of target molecules.

It relates more particularly to a kit for analysis of at least one target molecule, or of a family of target molecules, comprising at least two MIPs and at least one marker, as well as a method of analysis using said kit.

The detection and the determination of the concentration of an analyte present in a complex medium and/or present at trace levels in a sample, generally necessitate methods that, even today, still require laborious stages of purification and/or enrichment, as well as the use of very elaborate detectors, for instance mass spectrometers.

Furthermore, the methods used up to now for these treatments most often lack specificity. Moreover, the detection stage classically involves compounds that are chemically or mechanically unstable, which cannot be reused, and in addition it generally requires very long analysis times.

Accordingly, these methods frequently cause a loss of time and/or an increase in uncertainty of the result.

The analysis of such analytes can, however, be facilitated by carrying out, prior to the detection proper, a suitable pretreatment of the complex medium or of the sample. Thus, when the analyte is present in a complex medium such as urine, for example, which contains numerous additional entities that are likely to interfere with the measurement, or when the analyte is only present at trace levels in a sample and its detection is therefore difficult, it may be necessary to carry out preliminary extraction of said analyte.

Notably, the use of techniques of solid phase extraction (SPE) is already known, for concentrating analytes of interest contained in liquid samples, before analysis.

For this purpose, in particular the use of MIPs for the application of extractions of the SPE type is already known.

MIPs are in fact advantageous owing to their high specificity and their excellent chemical, mechanical and thermal stability. They are moreover known to be easily synthesizable and inexpensive if the template entity is available at low cost. As reported in Ohnmacht C. M., Chiel J. E., Hage D. S. Anal. Chem. 2006, 78, 7547-7556, the use of MIPs in a method of solid-phase extraction thus makes it possible to obtain advantageously, and in a single stage, an extract enriched in a given analyte.

Moreover, detection, or even determination of the concentration of an analyte by methods of analysis employing MIPs, is also already known.

Thus, MIPs can for example be used in tests of the radioimmunoassay type, in which said MIPs take the place of the antibodies and in which they can, at the same titer as the antibodies, be provided with markers that can be made to compete with analytes of interest.

Such methods of analysis are notably described in the review “Molecularly imprinted polymers in pseudoimmunoassay” by Richard J. Ansell, Journal of Chromatography B, 804 (2004) 151-155.

The use of MIPs in methods of analysis of this type offers numerous advantages, for example that of employing compounds that are more stable than antibodies, which can be used both in aqueous media and nonaqueous media, and do not require coupling of the analyte (in the case of small molecules), nor the use of laboratory animals for their production. This is why MIPs are gradually replacing antibodies in numerous areas of analysis.

Finally the use of several MIPs, which may be identical or different, in methods comprising a first pretreatment stage followed by a second stage of detection of analytes of interest, is also already known.

Thus, Chianella et al. have already described the use of one and the same MIP, on the one hand for concentrating a target molecule in the sample to be analyzed by SPE, and on the other hand for subsequent analysis of said target molecule. Detection of the target molecule is based in this document on the use of a piezoelectric sensor coated on the surface with the same MIP as that used for the stage of SPE concentration. Detection is then carried out by measuring the frequency, which is proportional to the mass of target molecule (Chianella I., Piletsky S. A., Tothill I. E., Chen B. Turner A. P. F., Biosensors and Bioelectronics, 2003, 18, 119-127).

The method described in this document is, however, difficult to implement for routine use, as it requires the use of piezoelectric sensors, which can only be used by highly trained personnel.

Document U.S. Pat. No. 6,461,873 teaches the application of two different MIPs for analysis of the caffeine contained in a drink. The method described in this document firstly comprises a first stage, during which the first MIP absorbs the interfering substances present in the drink to be analyzed. This first stage has the aim of improving the application of the second stage, dedicated for its part to the actual detection of the caffeine by using a second MIP, specifically absorbing the latter.

The method described in this document is, however, limited in its format and the physical nature of the device, to tests by chromatography on strips.

This format does not, moreover, permit subsequent enrichment of the analyte, assuming for example that it is present at trace levels in the analysis sample.

Furthermore, since the first MIP used is intended to recognize the interfering substances specifically, the pretreatment stage described in this document requires that the latter have been identified. Thus, the method according to this teaching therefore does not permit the analysis of analytes for application in media that are too complex.

P. S. Sharma et al. finally describe a method consisting of a first pretreatment stage of a solution containing creatinine by an SPE column containing an MIP specific to this molecule, followed by a stage of detection by differential-pulse cathodic stripping voltammetry implemented with a hanging mercury electrode modified by a second MIP identical to that employed during the first stage (P. S. Sharma, D. Lakshmi, B. B. Prasad, Chromatographia, 2007, 65, April (No. 7/8)).

This method seems, however, to be limited to the detection of electrosensitive molecules that are able to undergo oxidation or reduction.

Consequently, there is still a need for an analysis kit that can be used easily and quickly, for the detection, or additionally determination of the concentration, of an analyte present in a complex medium or present at trace levels in a sample.

In particular, there is still a need for an analysis kit that does not require cumbersome and/or expensive equipment such as, for example, equipment for chromatography (GC, LC, HPLC), piezoelectric sensors or equipment employing electrodes (such as in voltammetry techniques, notably of the anodic or cathodic stripping analysis type).

It is also desirable to have an analysis kit that is portable, that can be transported easily and used in the home, in a clinic, a doctor's office, outside of a hospital, in a factory or in the field.

There is also a need for an analysis kit that can be used for detecting analytes stripped of groups that are easily detectable, such as chromophores or fluorophores.

There is also still a need for an analysis kit with improved sensitivity, notably permitting the detection and determination of the concentration of analytes present at very low concentrations in a sample.

The inventors in fact discovered that the use of an analysis kit comprising at least two MIPs and at least one specific marker made it possible to meet these needs.

Thus, the present invention relates, according to one of its aspects, to a kit for analysis of at least one target molecule comprising at least a first and a second molecularly imprinted polymers which may be chemically identical or different, capable of interacting with the target molecule(s), and at least one marker, said marker being either a competitive marker or capable (i) of interacting with some or all of the recognition sites of the target molecule(s) of the second molecularly imprinted polymer and (ii) of being displaced from said recognition site(s) by the target molecule(s) on bringing said second molecularly imprinted polymer in contact with said target molecule(s), or an intrinsic marker or a constitutive unit of the second molecularly imprinted polymer and capable (i′) of interacting with all or some of the target molecule(s) when the latter interacts/interact with all or some of its/their recognition sites of said target molecule(s) and (ii′) of emitting, in consequence, a detectable signal.

The present invention also relates, according to one of its aspects, to a kit for analysis of at least one target molecule comprising at least a first and a second molecularly imprinted polymers that are chemically different, capable of interacting with the target molecule(s), and at least one marker, said marker being either a competitive marker or capable (i) of interacting with some or all of the recognition sites of the target molecule(s) of the second molecularly imprinted polymer and (ii) of being displaced from said recognition site(s) by the target molecule(s) on bringing said second molecularly imprinted polymer in contact with said target molecule(s), either an intrinsic marker or a constitutive unit of the second molecularly imprinted polymer and capable (i′) of interacting with all or some of the target molecule(s) when the latter interacts/interact with all or some of its/their recognition sites of said target molecule(s) and (ii′) of emitting, in consequence, a detectable signal.

Each of the variants described in detail below can also be applied to this particular embodiment.

The present invention also relates, according to another of its aspects, to a kit for analysis of at least one target molecule comprising at least a first and a second molecularly imprinted polymers that are chemically different, capable of interacting with the target molecule(s) and at least one marker, said marker being capable of emitting a detectable signal on bringing said second molecularly imprinted polymer in contact with said target molecule(s).

In other words, the object of the present invention is to provide users with an analysis kit offering the means for carrying out targeted analyses, and which can be varied both from the standpoint of its design and from the standpoint of its use, as will become clear on reading the following.

In particular, the vast range of MIPs that can be synthesized (in terms of polarity, hydrophobicity, site densities, magnetic and physicochemical properties, for example), the various conceivable conditions of use (in terms of pH, salinity, temperature, flow rate, pressure or polarity of the solvents, for example), the multiple markers that can be used, and the various supports available are the many parameters that it is possible to select, or adjust according to the needs of the user, and notably in relation to the nature of the target molecule(s), as well as according to the intended application (sensitivity of detection, selectivity limited for example to one particular target molecule or in contrast extended to a family of target molecules).

Depending on the intended application, it is thus in particular possible to vary the kit through the choice of “competitive” marker, namely capable (i) of interacting with some or all of the recognition sites of the target molecule(s) of the second molecularly imprinted polymer and (ii) of being displaced from said recognition site(s) by the target molecule(s) on bringing said second molecularly imprinted polymer in contact with said target molecule(s).

It is notably possible to adapt the latter's affinity for the recognition sites of the second MIP in relation to the required sensitivity and/or selectivity.

In fact, a competitive marker displaying strong affinity for these recognition sites can only be displaced by a particular target molecule, whereas a competitive marker displaying lower affinity can be displaced more easily, for example by a family of target molecules, or by a low concentration of a particular target molecule.

Moreover, it is also possible to vary the kit through the choice of “intrinsic” marker, namely the constitutive unit of the second molecularly imprinted polymer that is capable (i′) of interacting with all or some of the target molecule(s) when the latter interacts/interact with all or part of its/their recognition sites of said target molecule(s) and (ii′) of emitting, in consequence, a detectable signal.

Notably it is possible to make these variations by selecting said intrinsic marker according to the nature of its interactions with all or some of the target molecule(s) and/or the nature of the signal emitted, in relation to the required sensitivity and/or selectivity.

In particular it is possible to select an intrinsic marker that only displays interactions with all or part of a specific target molecule, or on the contrary an intrinsic marker displaying interactions with a family of target molecules. In this second case, it is also possible to select an intrinsic marker that emits different signals as a function of the target molecule of said family with which it interacts.

These various choices, and notably the adaptation of the relative affinity of the second MIP for the target molecule(s) and for the competitive marker, make it possible to provide a kit that can be varied, both in its specificity and in its sensitivity.

Said adaptation may in particular be advantageous for achieving, for example during a pretreatment stage, an optimal selectivity, namely such that the interfering entities present initially in the solution to be analyzed are, if necessary, removed, while preserving the target molecule(s) in the pretreated solution, and thus proceed to purification and optionally to enrichment of target molecule(s).

The invention also relates, according to another of its aspects, to a method of analysis of at least one target molecule comprising at least one stage of use of a kit according to the invention.

It also relates, according to another of its aspects, to the use of a kit according to the invention for the analysis of medicinal products, of pollutants, of chemical products, of contaminants, and the like.

The following meanings are adopted within the scope of the present invention:
“target molecule” or “analyte” means any entity that we wish to detect (i.e. whose presence we wish to demonstrate) and/or whose concentration in a given sample we wish to determine,
“marker” means any chemical entity, and for example any molecule similar in structure and/or affinity to the target molecule(s) or all or part of at least one constitutive unit of an MIP, and detectable by visible colorimetry, for example detectable with the naked eye, by radiochemistry, by nuclear medicine for example by scintigraphy, by imaging, by resonance (MRI), by X-rays, by diffusion of light, by mass spectrometry, by spectroscopy, for example by fluorescence or by visible UV, by infrared spectroscopy, by surface plasmon resonance spectroscopy, by chemiluminescence, by interference spectroscopy and refraction spectroscopy, by Raman scattering, by ultrasound, by radioactivity, by refractometry, by optical, piezoelectric, or acoustic detection, by electrochemistry, by conductivity, by measurement of pH or by biological means.
“signal” means a physical or chemical parameter that can be detected by any technique known by a person skilled in the art, notably by a technique mentioned above.

According to a preferred variant of the invention, the signal emitted by the marker is detected by visible colorimetry, by fluorescence or by UV, and preferably by the naked eye.

As an example of signal according to the invention, we may notably mention an absorption or emission wavelength detectable by visible colorimetry or by spectroscopy or by fluorescence, a color change detectable with the naked eye, a refractive index detectable by surface plasmon resonance spectroscopy, a luminosity detectable by diffusion of light, a mass detectable by a piezoelectric sensor, beta emission detectable by a scintillation counter, an oxidation potential detectable by electrochemistry.
“recognition site” means the cavity in the matrix of the MIP that is effectively involved in the recognition of the target molecule(s),
“complex medium” means a medium comprising, in addition to the target molecule(s), one or more other additional entities, at least some of which are capable of interfering with said target molecule(s) during at least one of the stages of the analysis (pretreatment stage and/or detection stage), and of disturbing the interpretation of the results.

As examples of complex media according to the invention, we may notably mention body fluids such as blood, plasma, saliva, urine, bile, tears, mother's milk, or culture media, cellular lysates, plant extracts, foodstuffs, environmental media (soil, water, air), drinks such as wine, milk, fruit juice, beer, or gaseous media.
“trace level” means any concentration between 1 ng/L and 10 mg/L, notably between 5 ng/L and 1 μg/L, preferably between 10 ng/L and 500 ng/L.
“interaction” occurring between the target molecule(s) and a recognition site, between a competitive marker and a recognition site or between an intrinsic marker and the target molecule(s), means the formation of weak bonds (for example of the Van der Waals type, hydrogen bonds, pi donor/pi acceptor bonds, or hydrophobic interactions) and/or of strong bonds (for example such as ionic bonds, covalent bonds or ionic-covalent bonds, coordination bonds, and dative bonds).

Within the scope of the present invention, the interactions between the target molecule(s) and a recognition site, between a competitive marker and a recognition site or between an intrinsic marker and the target molecule(s) can be identical or different within one and the same MIP.

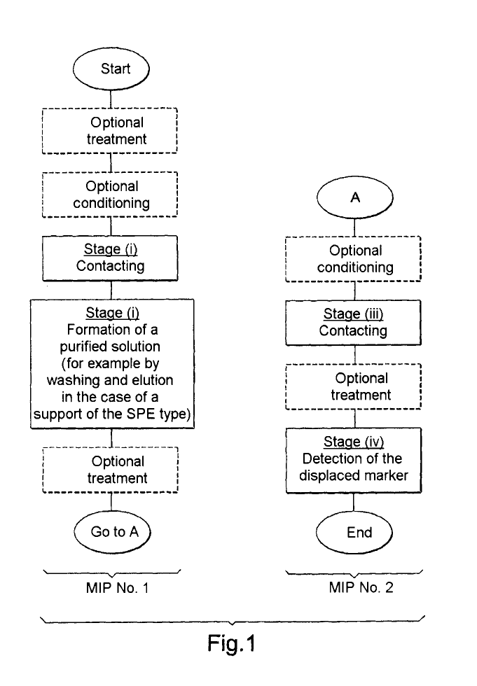

FIG. 1 represents, in the form of a block diagram, the various stages of the method of analysis according to the invention.

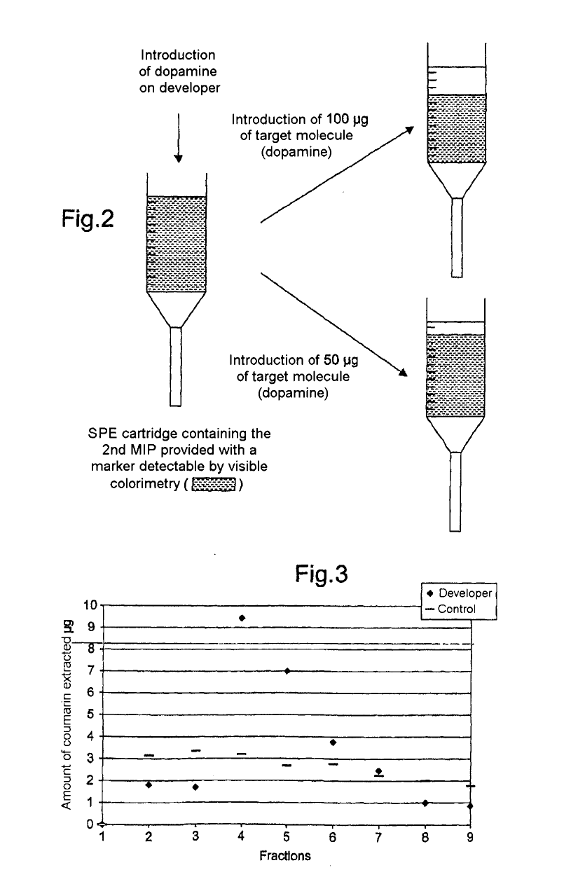

FIG. 2 illustrates the application of the detection stage of the method according to the invention, according to the particular embodiment in which the second molecularly imprinted polymer is packaged in an SPE cartridge and the target molecule is dopamine and the marker is detectable with the naked eye, in accordance with the examples.

FIG. 3 is a graph showing the amounts of 6,7-hydroxy-4-trifluoromethyl coumarin obtained in the various 1-ml fractions of MeOH percolated through the “control” and “developer” SPE cartridges described in example 6, test B.

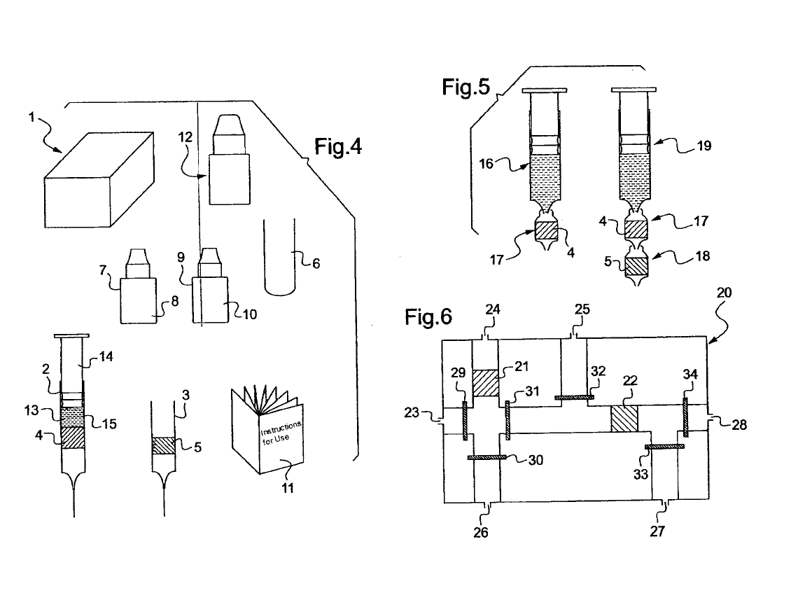

FIG. 4 shows a schematic, perspective view of an analysis kit according to the invention,

FIG. 5 shows part of a variant of an analysis kit according to FIG. 4.

FIG. 6 illustrates an embodiment in which the MIPs are integrated in a block system.

It is understood that the MIPs of the kit according to the invention can be intended for recognizing several target molecules depending on the intended use. Thus, unless stated otherwise, the invention is not limited just to the embodiments where the properties of recognition only aim at a single target molecule.

In particular, according to one embodiment, the kit according to the invention can be intended for the analysis of a family of target molecules, i.e. for the analysis of a set of different molecules that can be detected by one and the same recognition site of an MIP and possessing for example structural similarity and/or a given, common arrangement of functional groups.

Of course, depending on the intended application, and notably depending on the required selectivity, the family of target molecules that can be analyzed can comprise a broader or narrower set of different molecules.

Analysis Kit

The analysis kit as claimed in the invention comprises at least two MIPs, which may be chemically identical or different, having at least some recognition sites capable of interacting with the target molecule(s) (also called “recognition sites of the target molecule(s)”).

In the sense of the invention, the term “kit” denotes a packaging assembly, in which said at least two MIPs are packaged separately from one another, for example in two compartments or on two separate supports as defined below, for example on SPE cartridges. Said supports, and for example said cartridges, can then be arranged within one and the same block system, as illustrated for example in FIG. 6.

At least one molecularly imprinted polymer (called “first MIP” hereinafter) can be intended for a stage of pretreatment of the sample, for example for extraction of the target molecule(s), by molecular recognition of the target molecule(s).

“Extraction of the target molecule(s) by molecular recognition of the target molecule(s)” means, in the sense of the invention, a stage during which the interaction of the target molecule(s) with the recognition sites of an MIP is sufficient to lead to the formation of a complex composed of the MIP provided, in all or some of its recognition sites, with said target molecule(s).

In consequence, the recognition sites of the first MIP, and notably its recognition sites of the target molecule(s), can initially be partly, or even preferably completely, vacant.

Thus, according to one embodiment of the invention, all of the recognition sites of the target molecule(s) of the first MIP of the kit according to the invention are vacant.

According to another embodiment of the invention, all of the recognition sites of the first MIP of the kit according to the invention are vacant.

The first MIP can thus make it possible, according to one embodiment of the invention and after release of the extracted target molecule(s), to obtain a solution that is purified, or enriched in the target molecule(s).

“Release of the extracted target molecule(s)” means, in the sense of the invention, a stage during which the complex formed during extraction of the target molecule(s) by molecular recognition of the target molecule(s) undergoes dissociation, for example as a result of a change in the conditions of pH, of salinity, of temperature, of flow rate, of pressure, or of polarity of the solvents, leading to the presence of the target molecule(s) in free form in solution.

At least one other molecularly imprinted polymer (called “second MIP” hereinafter), in chemical nature identical to or different from the first molecularly imprinted polymer, can, either when it is provided in all or some of its recognition sites of the target molecule(s) with at least one competitive marker, or when it is constituted of at least one intrinsic marker, as defined below, be intended for the detection, or determination of the concentration, of the target molecule or molecules, as is described in more detail below.

The marker, the signal or a variation of signal emitted by the marker can notably be detected, for example after salting-out or as a result of an interaction with the target molecule, by colorimetry, visible for example to the naked eye, by radiochemistry, by nuclear medicine for example by scintigraphy, by imaging, by resonance (MRI), by X-rays, by diffusion of light, by mass spectrometry, by spectroscopy, for example by fluorescence or by visible UV, by infrared spectroscopy, by surface plasmon resonance spectroscopy, by chemiluminescence, by interference spectroscopy and refraction spectroscopy, by Raman scattering, by ultrasound, by radioactivity, by refractometry, by optical, piezoelectric, or acoustic detection, by electrochemistry, by conductivity, by pH measurement, or by biological means, and preferably by the naked eye.

According to a preferred variant of the invention, the marker, the signal or the variation of signal emitted by the marker is detected by visible colorimetry, by fluorescence or by UV, and preferably with the naked eye.

According to a preferred embodiment of the invention, the second molecularly imprinted polymer can be of different chemical nature than the first molecularly imprinted polymer.

It may in fact be advantageous to select the first and second MIPs in such a way that they exhibit different affinities for the target molecule(s), and in particular affinities that are optimized in relation to their respective conditions of use. Said optimization of the recognition of the target molecule(s) for each stage of the analysis may thus make it possible for example for the target molecule to be targeted with maximum efficiency during the stages of pretreatment and detection.

As explained in more detail below, selection of two MIPs of different chemical nature is also useful in that it makes it possible to use the purified solution obtained at the end of the first stage for application of the detection stage.

Competitive Marker

According to a first embodiment, the marker can be capable (i) of interacting with some or all of the recognition sites of the target molecule(s) of the second molecularly imprinted polymer and (ii) of being displaced from said recognition site(s) by the target molecule(s) on bringing said second molecularly imprinted polymer in contact with said target molecule(s).

Said marker is designated as “first type of marker” or “competitive marker” hereinafter.

When the first and the second MIPs are chemically identical, it is to be understood, within the scope of the present invention, that they must be packaged separately.

However, according to a first embodiment of the invention, at least the competitive marker and the second molecularly imprinted polymer can be packaged separately, for example in two separate compartments.

In this case, the user brings the marker in contact with the second MIP, so that said marker interacts with the recognition sites of the target molecule(s) of said second MIP, prior to bringing the purified solution, optionally enriched in target molecule(s), in contact with the second MIP.

Alternatively, at least the competitive marker and the second molecularly imprinted polymer can be packaged together in such a way that the second molecularly imprinted polymer is provided, in all or some of its recognition sites of the target molecule(s), with said marker.

In both of these cases, said marker must be able to interact with some or all of the recognition sites of the target molecule(s) of the second MIP, and also be displaceable by the target molecule(s) from all or some of these sites when the second MIP is put in contact with the target molecule(s).

Of course, the competitive marker can only be displaced from these sites by the target molecule(s) if the conditions of bringing the second MIP, provided with said marker, in contact with said target molecule(s) are favorable to interaction of said target molecule(s) with the recognition sites of said second MIP.

The second MIP can also have, as well as the recognition sites of the target molecule(s), various other recognition sites, each of which is also able to interact with at least one competitive marker according to the invention. These other recognition sites can thus make it possible to detect, or determine the concentration of, other types of target molecules in the same conditions of use or in different conditions of use.

The competitive marker must therefore have lower affinity for these recognition sites than the target molecule(s), in the conditions of use of the second MIP.

Thus, when the second MIP according to the invention is provided, in all or some of its recognition sites of the target molecule(s), with at least one competitive marker, and is then put in contact with a solution containing the target molecule(s), said marker must be displaced from the recognition sites of said second MIP by the target molecule(s) and thus be substituted with said target molecule(s): this is called competitive displacement of the marker by the target molecule(s).

This competitive displacement leads to the salting-out (i.e. release in the analysis medium, in free form) of said marker, which can be detected by any method suitable for detection of the marker.

The present invention includes the case when detection is different depending on whether the competitive marker is in free form, for example after salting-out, or is still present in the recognition sites of the MIP.

According to one embodiment, each recognition site of the target molecule(s) of the second molecularly imprinted polymer can interact with a single competitive marker, notably a single molecule of competitive marker.

According to another embodiment, each recognition site of the target molecule(s) of the second molecularly imprinted polymer can interact with several competitive markers, notably several molecules of competitive marker.

According to one embodiment of the invention, the first and second molecularly imprinted polymers can be identical in terms of chemical nature. In particular, when the second molecularly imprinted polymer and the first type of marker or competitive marker are packaged together in such a way that the second molecularly imprinted polymer is provided, in all or some of its recognition sites of the target molecule(s), with at least one marker, the first and second molecularly imprinted polymers may differ solely by the presence of said marker in some or all of the recognition sites of the target molecule(s) of the second molecularly imprinted polymer.

According to a preferred embodiment of the invention, the first and second molecularly imprinted polymers can be of different chemical nature, regardless of whether or not at least one competitive marker is present in some or all of the recognition sites of the target molecule(s) of said second molecularly imprinted polymer.

Intrinsic Marker

According to a second embodiment, the marker can be a constitutive unit of the second molecularly imprinted polymer and be capable (i′) of interacting with all or some of the target molecule(s) when the latter interacts/interact with all or some of its/their recognition sites of said target molecule(s) and (ii′) of emitting, in consequence, a detectable signal.

Such a marker is designated as “second type of marker” or “intrinsic marker” hereinafter.

“Constitutive of the second molecularly imprinted polymer” is understood, in the sense of the invention, as forming part of the chemical constitution of said molecularly imprinted polymer. In other words, a second molecularly imprinted polymer according to this second embodiment is formed partly, but not necessarily exclusively, of such a unit.

According to one embodiment, the intrinsic marker can be partly constitutive of the recognition sites of the target molecule(s) of the second molecularly imprinted polymer.

According to another embodiment, the intrinsic marker need not be constitutive of said recognition sites of the target molecule(s) of the second molecularly imprinted polymer, but be located near the latter.

Ways of preparing said molecularly imprinted polymers are described below, for purposes of illustration.

In the sense of the invention, when it is a question of interaction of an intrinsic marker with all or some of the target molecule(s) in the conditions indicated previously, it is to be understood not only as direct interaction between said marker and all or some of said target molecule(s) causing the emission of a detectable signal by said marker, but also any indirect interaction on said marker that can be induced by an interaction between another chemical entity and all or some of said target molecule(s).

As an example of such indirect interactions, we may notably mention any interaction between on the one hand a group, for example a functional group, bound covalently to said marker, and on the other hand all or some of the target molecule(s), provided that this interaction leads to the emission of a detectable signal by said marker.

Said group can be bound directly to the marker by a covalent bond or can be separated from said marker by a spacer (“linker”), as described below.

The indirect interactions then occurring between the marker and the target molecule may be due to a change in the environment, for example the electronic environment, of the marker, induced in particular by said group.

Still according to this second embodiment, the interactions between the intrinsic marker and all or some of the target molecule(s) occur when the latter interact with some or all of the recognition sites of said target molecule(s) of the second molecularly imprinted polymer.

Moreover, this chain of interactions should, according to the invention, cause the emission of a detectable signal by the marker.

According to one embodiment, a variation of the signal emitted by the marker can also be detected, in particular a variation between on the one hand the signal emitted by the marker before interaction with all or some of the target molecule(s) and on the other hand the signal emitted by the marker in the course of said interaction.

According to the invention, the second molecularly imprinted polymer constituted of at least one intrinsic marker can result from the copolymerization of at least one monomer constituted wholly or partly of at least said marker.

Such monomers can notably be obtained by chemically modifying a marker by incorporating a polymerizable unit in it, if necessary separated from said marker by a spacer.

Techniques for synthesizing monomers of this type are already known from the prior art, and form part of the general knowledge of a person skilled in the art.

As an example, notably the synthesis of 4-trifluoroacetyl-4′-[N-(methacryloxyethyl)-N-(ethyl)amino]-azobenzene from 4-N,N-dioctylamino-4′-trifluoroacetyl-azobenzene is already known from A. Gräte et al. (A. Grate, K. Haupt, G. J. Mohr, Analytica Chimica Acta, 565, 2006, 42-47).

A monomer modified in this way can then participate in the synthesis of the second MIP, and the unpolymerized part of this monomer can thus become a constitutive unit of the latter, as indicated below.

According to a preferred embodiment, each recognition site of the target molecule(s) of the second molecularly imprinted polymer can be constituted of, or have in its vicinity, a single intrinsic marker, notably a single molecule of intrinsic marker.

According to another embodiment, each recognition site of the target molecule(s) of the second molecularly imprinted polymer can be constituted of, or have in its vicinity, several intrinsic markers, notably several molecules of intrinsic marker.

Application of the Kit

The first and second MIPs can be used on any appropriate support.

“Support” means, very broadly, in the sense of the invention, any solid, flexible or rigid substrate, on or in which the MIPs can be bound, cemented, deposited, synthesized in-situ, filled and/or packaged.

The supports that can be used according to the invention can be of any nature, for example of biological, nonbiological, organic, or inorganic nature, or a combination thereof. They can be in any form, and notably can be in the form of particles, gels, sheets, tubes, spheres, capillaries, dots, films, wells, of any size and of any shape.

According to one embodiment, the supports can more particularly be in the form of films.

The MIPs can for example be in the form of particles of uniform size, notably between 10 nm and 10 mm, for example between 100 nm and 1 mm, notably between 1 μm and 100 μm, preferably between 25 and 45 μm, and can then be packaged in the form of a cartridge.

Generally, the first and second MIPs can for example be used on or in a support selected from an SPE cartridge, a multiwell plate, for example a 96-well plate, a patch, a tea bag, a microtube, an HPLC column, a strip, for example strips with a format similar to that of strips of pH paper, chips, laminas, silica plates, thin layers, a porous surface, a nonporous surface, a microfluidic system.

According to one embodiment, the method of analysis according to the invention can optionally comprise in addition a stage of packaging of the first molecularly imprinted polymer prior to the pretreatment stages (i) and (ii) described below and/or a stage of packaging of the second molecularly imprinted polymer prior to the detection stages (iii) and (iv) described below.

According to one embodiment of the invention, the first molecularly imprinted polymer can be used in an extraction column, for example an SPE cartridge.

According to another embodiment of the invention, the second molecularly imprinted polymer can be used in an SPE cartridge, optionally graduated. FIG. 2 illustrates this aspect of the invention when the marker is detectable with the naked eye.

The kit according to the invention can for example be in the form of a packaging assembly, comprising at least one SPE cartridge containing the first MIP which will permit loading, washing and then elution for recovering the target molecule(s) in free form and at least one SPE cartridge containing the second MIP with either at least one type of recognition sites containing at least one competitive marker as described previously that can be displaced by the target molecule(s), or at least one intrinsic marker as described previously that is able to emit, as a result of its interaction with all or some of the target molecule(s), a detectable signal.

Owing to the great variety of choice of the nature of the two MIPs, the nature of the marker, or the conditions of use, it is possible to vary the analysis kit as claimed in the invention very extensively, and adapt it to the intended applications.

Advantageously, and as stated previously, the MIPs can notably be optimized so as to obtain the best properties of recognition and detection for each of the stages of the analysis (pretreatment and detection), according to the affinity of the target molecule or molecules and, if applicable, of the competitive marker, against them or according to the interaction between the intrinsic marker and said target molecule(s).

In the sense of the invention, the term “affinity” denotes the ability of an entity (for example a target molecule or a marker as defined previously) to interact with a recognition site of an MIP. Strong affinity thus means, in the sense of the invention, that said entity has considerable ability to interact with at least one recognition site of an MIP.

As stated previously, interaction means, in the sense of the invention, the formation of weak bonds (for example such as Van der Waals bonds, hydrogen bonds, pi donor/pi acceptor bonds, or hydrophobic interactions) and/or formation of strong bonds (for example such as ionic bonds, covalent bonds, or ionic-covalent bonds, coordination bonds and dative bonds).

It is to be understood that the interaction that occurs during preparation of the MIP may be different from that which occurs during its use.

For example, there may be an unstable covalent interaction during its manufacture and an interaction of the ionic and hydrogen type during its use.

An entity's affinity for a recognition site of an MIP can be evaluated by experimentally measuring the capacity factor of this site with respect to this entity, for example by carrying out a chromatographic analysis of the entity whose affinity we wish to determine through a stationary phase comprising at least the recognition sites of the MIP in respect of which we wish to determine the affinity of said entity.

The capacity factor k' of these recognition sites corresponds according to this protocol to the ratio of the time spent by this entity in the stationary phase, relative to the time spent by this same entity in the mobile phase.

It can be determined experimentally from the ratio:

$${k}^{′} = \frac{{t}_{r} - {t}_{m}}{{t}_{m}} ,$$

in which tᵣ denotes the retention time, and tₘ the dead time.

However, the capacity factor takes account not only of the specific interactions of molecular recognition between the target molecule(s) and the recognition sites of the MIP, but also of the nonspecific interactions between said target molecule(s) and the material of which said MIP is constituted.

Thus, often the imprinting factor (IF) is used, which corresponds to the ratio of the capacity factor of the MIP (i) to the capacity factor of the material of which said MIP is constituted (i), also called “unimprinted polymer”

$$IF = \frac{{k}_{MIP}^{′} ( i )}{{k}_{unimprinted polymer}^{′} ( i )}$$

The imprinting factor thus takes into account the effectiveness of an MIP in terms of molecular recognition, independently of the chemical nature of its polymer matrix.

Quite clearly, the affinity depends both on the entity and on the MIP, but it also varies in relation to the conditions of use.

Thus, it can vary in particular with the solvating medium, and may depend notably on the conditions of pH, salinity of the medium, temperature, flow rate, pressure, as well as the polarity of the solvent.

It is nevertheless possible to compare for example the affinities of two different entities for one and the same MIP in identical conditions of use, or the different affinities of one and the same entity for one and the same MIP in varied conditions of use.

The affinity can also be adapted, according to the intended use, during preparation of the first and second MIPs, in particular by varying the conditions of synthesis (nature of the solvent, temperature, concentration of the reactants, nature of the primer, etc.), the template entities, the initial monomers or the crosslinking agent used for making the MIP.

The polymerization stage of the MIP around a template entity makes use of techniques that are known per se by a person skilled in the art. We may thus refer to the article by Peter A. G. Cormack et al., Journal of Chromatography B, 804 (2004) 173-182, which presents a review of the techniques available regarding aspects of the polymerization of MIPs. The contents of this article are incorporated here by reference.

In general, these MIPs are obtained by copolymerizing the monomers and crosslinking agent(s) in the presence of an entity whose imprint is to be formed. The monomers become arranged specifically around this entity, also called “template entity”, by strong or weak interactions, and are then polymerized generally in the presence of a high level of crosslinking agent. After polymerization, the entity is removed from the polymer material and thus leaves its molecular imprint in cavities within the material, which constitute true synthetic receptors comparable to biological receptors of the antibody type.

The template entity can be identical to or different from the target molecule. Thus, document WO 07/004,197 notably describes the use of a template entity of polymeric nature, different from the target molecule(s), for the synthesis of MIPs.

More precisely, there are two possible approaches for making MIPs: the covalent approach developed by Wulff in document U.S. Pat. No. 4,127,730 and the noncovalent approach developed by Mosbach in document U.S. Pat. No. 5,110,833.

In the covalent approach, the interactions between the monomers and the template entity are of the nature of labile covalent bonds. In this case, after extraction of the template entity by rupture of the covalent bond, the recognition of target molecules is also effected by the formation of a covalent bond between the imprint and the target molecule in question.

In the noncovalent approach of Mosbach, the interactions between the monomers and the template entity are weak bonds of the nature of hydrogen bonds, pi donor/pi acceptor bonds, Van der Waals bonds or hydrophobic interactions. After extraction of the template entity, the recognition of target molecules is also effected by noncovalent interactions between the imprint and the target molecule.

These two approaches can be combined.

Thus, it is possible to use the first approach of the covalent type for preparation of the MIP and the second approach to obtain recognition by noncovalent interactions, as is disclosed for example in M. J. Whitcombe et al. “A New Method for the Introduction of Recognition Site Functionality into Polymers prepared by Molecular Imprinting: Synthesis and Characterization of Polymeric Receptors for Cholesterol” J. Am. Chem. Soc., 1995, 117, 7105-7111.

It is also possible to use the first and second approaches for preparation of the MIP, as well as for obtaining recognition by covalent and noncovalent interactions simultaneously for one and the same target molecule. Thus, the interaction develops at least in two separate sites of the recognition site, as disclosed for example in Wulff G. et al. Macromol. Chem. Phys. 1989, 190, 1717 and 1727.

The MIP or more precisely the matrix of which it is constituted can thus be formed by radical copolymerization. Vinylic monomers, monomers derived from styrene, from methacrylic acid, are monomers that are particularly suitable for this technique. Any initiator can be used, such as azobisisobutyronitrile (AIBN).

When the second MIP comprises at least one intrinsic marker as described previously, it can notably be obtained by copolymerization of at least one particular monomer constituted of at least one polymerizable unit, of said marker and, if necessary, of a spacer.

The techniques of modification of a marker, for example to graft a polymerizable unit on it, if necessary via a spacer, are well known by a person skilled in the art.

As monomers of this type, we may notably mention (E)-1-(2-methacryloxyethyl)-3-[4-(4-nitrophenyldiazenylphenyl]thiourea.

According to one embodiment, notably when the target molecule is ochratoxin, the first and/or the second molecularly imprinted polymer, and in particular the second molecularly imprinted polymer, can employ at least (E)-1-(2-methacryloxyethyl)-3-[4-(4-nitrophenyldiazenylphenyl]thiourea during their polymerization stage.

The MIP can also be formed by radical copolymerization, with the aim of varying the properties of the polymer. We may mention for example the methyl methacrylate/butyl methacrylate copolymer.

The MIP can be formed from crosslinked polymers or copolymers. Depending on the degree of crosslinking, we use the terms branched polymer or branched copolymer, macroscopic networks or microgels.

Among other monomers that can be used for synthesis of the MIPs, we may mention:
acidic monomers: methacrylic acid (MAA), p-vinylbenzoic acid, acrylic acid (AA), itaconic acid, 2-(trifluoromethyl)-acrylic acid (TFMAA), acrylamido-(2-methyl)-propane sulfonic acid (AMPSA), 2-carboxyethyl acrylate,
basic monomers: 4-vinylpyridine (4-VP), 2-vinylpyridine (2-VP), 4-(5)-vinylimidazole, 1-vinylimidazole, allylamine, N,N′-diethyl aminoethyl methacrylamide (DEAEM), N-(2-aminoethyl)-methacrylamide, N,N′-diethyl-4-styrylamidine, N,N,N-trimethyl aminoethylmethacrylate, N-vinylpyrrolidone (NVP), urocanic ethyl ester,
neutral monomers: acrylamide, methacrylamide, 2-hydroxyethyl methacrylate (2-HEMA), trans-3-(3-pyridyl)-acrylic acid, acrylonitrile (AN), methyl methacrylate (MMA), styrene, ethylstyrene.

It is within the general competence of a person skilled in the art to prepare the first and second MIPs having the properties required according to the intended application.

It is thus also possible to use, for the synthesis of the first and/or second MIPs, specific monomers according to the target molecule(s) required, and in particular, at least partly, monomers derived from a target molecule, thus partly performing the role of the polymer of the matrix and partly the role of the template entity. In other words, a proportion of these monomers, once polymerized, are intended to be removed in order to give rise to the recognition sites.

As an example of such monomers, we may notably mention 5-[2-(N-tert-butoxycarbonyl)ethylamino]-2-(4-vinylphenyl)benzo[1,3,2]dioxoborole, which is derived from N-tert-butoxycarbonyl-3,4-dihydroxyphenylethylamine, itself derived from dopamine.

According to one embodiment of the invention, and notably when the target molecule is dopamine, the first and/or second molecularly imprinted polymers can employ at least 5-[2-(N-tert-butoxycarbonypethylamino]-2-(4-vinylphenyl)benzo[1,3,2]dioxoborole during their polymerization stage.

According to this embodiment of the invention, it/they can in particular be obtained by copolymerization of at least 5-[2-(N-tert-butoxycarbonyl)ethylamino]-2-[4-vinylphenyl]benzo[1,3,2]dioxoborole and at least 2-carboxyethyl acrylate, with for example at least divinylbenzene or ethylene glycol dimethacrylate.

Method of Analysis

Reference may be made to FIG. 1, showing the arrangement of the various stages of the method according to the invention.

The present invention also relates, according to another of its aspects, to a method of analysis of at least one target molecule comprising at least one stage of use of a kit according to the invention.

The target molecule(s) can for example be present in a solution, and in particular can be present in a complex medium, or can be present at trace levels in a sample.

Notably it can be a method of analysis of at least one molecule that may be present in a solution, comprising at least:

(i) a stage of contacting said solution with a first molecularly imprinted polymer that is able to interact with the target molecule(s) in conditions suitable for extraction of the target molecule(s),

(ii) the formation of a solution that has been purified, and optionally enriched in target molecule(s), starting from the target molecule(s) isolated in stage (i),

(iii) a stage of contacting said purified solution, optionally enriched in target molecule(s), with a second molecularly imprinted polymer, also able to interact with said target molecule(s), and either provided, in all or some of its recognition sites of the target molecule(s), with at least one competitive marker or marker that can be displaced from said recognition site(s) by the target molecule(s) in the conditions of carrying out said stage (iii), or constituted of at least one intrinsic marker or marker that is able to interact with all or some of said target molecule(s) when the latter interacts/interact with all or part of its/their recognition sites of said target molecule(s) and emit in consequence a detectable signal and

(iv) a stage of qualitative, quantitative and/or semiquantitative detection of the target molecule(s), through the detection either of the competitive marker thus displaced, or of the signal thus emitted by the intrinsic marker.

As stated previously, the competitive marker and the second MIP can be packaged separately in two separate compartments, or can be packaged together in such a way that the second MIP is provided, in all or some of its recognition sites of the target molecule(s), with said marker.

Thus, according to one variant of application, the method according to the invention can additionally comprise, prior to stage (iii), a stage of contacting the second MIP with at least one competitive marker according to the invention. The conditions will then advantageously be adapted to the interaction of said marker(s) with the recognition sites of the target molecule(s) of the second MIP.

### Pretreatment Stage

According to one embodiment of the invention, stage (i) can be a stage of pretreatment of the solution containing the target molecule(s), and for example can be used for extraction of the target molecule(s), so as to obtain in consequence a purified solution, optionally enriched in target molecule(s).

It is followed by a stage (ii) of release of the target molecule(s) extracted by the first MIP, so as to lead to the formation of a purified solution, optionally enriched in target molecule(s), starting from the target molecule(s) isolated in stage (i).

Stage (ii) can for example be carried out by bringing the target molecule(s) isolated in stage (i) in contact with a medium that promotes rupture of their interaction with the first molecularly imprinted polymer.

Thus, when the solution to be analyzed is for example a complex medium, said stage (i) of the pretreatment makes it possible to remove the supplementary entities, different from the target molecule(s), and capable of interfering with the application of stage (iii).

As a variant, when the solution to be analyzed is a sample containing the target molecule(s) at trace levels, stage (i) of the pretreatment makes it possible to obtain, after release of the target molecule(s) extracted by the first MIP, a pretreated solution enriched in target molecule(s), namely in which the target molecule(s) is/are present at higher contents in comparison with the initial solution that has not undergone stage (i).

The degree of enrichment obtained at the end of stage (i) can for example be between 2 and 1000, notably between 5 and 100.

According to one embodiment of the invention, it can notably be a stage of solid-phase extraction (SPE).

A procedure for solid-phase extraction generally comprises three or four stages. The first is the packaging of the adsorbent (comprising according to the invention at least one first MIP contained in the extraction cartridge, which makes it possible to wet the support by solvating the functional groups present on its surface.

During the second stage, we proceed to the percolation of the solution to be treated on the first MIP, in such a way that entities having no affinity for the latter are not retained.

In contrast, the target molecule(s), and optionally other entities displaying strong affinity for the adsorbent, remain on the support at the end of this stage.

An additional washing stage can be carried out in order to remove the entities that are weakly retained by the support, by means of a solvent with suitable eluting power for eluting these entities while keeping the target molecule(s) on the support.

Finally we proceed to elution of the target molecule(s) by passage of a solvent specifically selected to permit rupture of the recognition interactions that developed between the target molecule(s) and the first MIP while avoiding elution of the interfering entities strongly retained on the support, so as to release the extracted target molecule(s).

At the end of this process of extraction and release, we therefore obtain a solution that has been purified, and optionally enriched in target molecule(s). As stated previously, this pretreated solution can be used as such for the third stage (iii), or can undergo further pretreatment, before it is brought in contact with the second MIP.

According to a preferred variant of the invention, said purified solution, optionally enriched in target molecule(s), is used as such for the third stage (iii), i.e. without undergoing prior treatment.

Typically, the solvents used during a solid-phase extraction can be organic solvents, for example acetonitrile, methanol, dichloromethane, aqueous solvents for example water, buffer solutions, solvents that can be used in a mixture and with different conditions of salinity, of pH, and of polarity.

It is known that the target molecule will be retained even more specifically in an MIP if the same type of interactions develop there as those that developed during synthesis.

Thus, according to one embodiment of the invention, stage (i) can be carried out using a solvent that is identical or similar to the solvent used during synthesis of the first MIP. Other types of pretreatment stages can be considered, for example solid phase micro-extraction (SPME), solid phase dynamic extraction (SPDE), stir bar sorption extraction (SBSE), capillaries, strips, chips.

The choice and the synthesis of the MIP, as well as the adaptation of the conditions of use, are within the general knowledge of a person skilled in the art.

Detection Stage

According to the invention, stages (iii) and (iv) of detection of the target molecule(s) are carried out after the pretreatment stages (i) and (ii) described above.

As stated previously, the application of a detection stage according to the invention advantageously makes possible the simple detection of the analytes that do not have easily detectable groups.

The purified solution, optionally enriched in target molecule(s), obtained at the end of stage (ii) can be contacted directly with the second MIP that is provided, in all or some of its recognition sites for the target molecule(s), with at least one marker as described previously. Alternatively it can be treated prior to contacting with said second MIP, for example so as to modify its pH, the concentration of target molecule(s), its salinity, or its polarity.

When “conditions” are referred to in the following paragraphs, this is to be understood as the conditions of pH, of concentration of target molecule(s), of salinity, or of polarity.

Thus, according to a preferred variant of the invention, the solution obtained at the end of stage (ii) can be contacted directly with the second MIP, said second MIP being either provided, in all or some of its recognition sites, with at least one competitive marker as described previously, or constituted of at least one intrinsic marker as described previously.

In this case, the first and second MIPs are of different chemical nature, and the competitive marker is selected in such a way that it is able to be displaced by the target molecule(s), in the conditions employed during the stage of release of the target molecule(s) extracted by the first MIP.

Alternatively, the intrinsic marker is selected in such a way that it is able to interact with all or some of the target molecule(s) when the latter interacts/interact with some or all of the recognition sites of said target molecule(s) of the second MIP, and emit in consequence a detectable signal in the conditions employed during the stage of release of the target molecule(s) extracted by the first MIP.

This first variant of application notably makes it possible to limit the number of stages required for carrying out the method according to the invention, and therefore to limit the number of manipulations and thus generally facilitate the application of the method according to the invention.

According to another variant of application, the solution obtained at the end of stage (ii) can be treated before it is brought in contact either with the second MIP that is provided, in all or some of its recognition sites for the target molecule(s), with at least one competitive marker as described previously, or with the second MIP constituted of at least one intrinsic marker as described previously.

In this case, we can use either first and second MIPs that are identical in terms of chemical nature, differing for example only by the presence of at least one marker, for example a competitive marker in some or all of the recognition sites of the target molecule(s) of the second MIP, or first and second MIPs of different chemical nature, in such a way that the marker is either able to be displaced by the target molecule(s), in the conditions employed during the stage of release of the target molecule(s) extracted by the first MIP (in the case of a competitive marker), or is able to interact with the target molecule(s) and emit in consequence a detectable signal (in the case of an intrinsic marker).

When the first and second MIPs are identical in terms of chemical nature, the solution enriched in target molecule(s) obtained at the end of stage (ii) can notably, according to this second variant of application, be treated so as to obtain conditions of pH, of concentration of target molecule(s), of salinity and/or of polarity approaching those employed during stage (i) for the extraction of the target molecule(s).

In contrast, when the first and second MIPs are of different chemical nature, this additional stage of treatment can for example consist of waiting for the best conditions of use required for the application of stage (iii).

Competitive Marker

According to a first embodiment, stage (iii) is based on a test of competition between the target molecule(s) and the competitive marker with respect to the recognition sites of a second MIP.

Tests of competition between an analyte and a marker adsorbed on MIPs have already been described in the literature. Thus, reference may be made for example to the review of Richard J. Ansell, Journal of Chromatography B, 804 (2004) 151-155, already cited in the preamble, which lists the different ways of using MIPs in such tests, notably in radioimmunoassay.

As noted in the description of the kit according to the invention, the competitive marker used in stage (iii) must have lower affinity for the recognition sites of the target molecule(s) of the second MIP than the target molecule(s), in the conditions of use of the second MIP in this stage.

Owing to this difference in affinity, bringing the second MIP, provided in all or some of its recognition sites of the target molecule(s) with said marker, with the pretreated solution that has been purified and optionally enriched in target molecule(s) will lead to displacement of the marker by the target molecule(s), which will thus be replaced in the second MIP by the target molecule(s).

Detection of the target molecule(s) in stage (iii) is thus based on a shift of the equilibrium between on the one hand the second MIP provided with said marker(s) and on the other hand the second MIP provided with the target molecule(s), the formation of this last-mentioned complex being favored.

Once again, the second MIP can be adapted according to the knowledge of a person skilled in the art, in relation to the target molecule(s) and/or the marker and/or the conditions of use and/or the intended application.

The choice of the competitive marker can also be adapted according to the general knowledge of a person skilled in the art.

For example, when the molecule is dopamine, and when the second MIP is used in methanol, the competitive marker can notably be 6,7-hydroxy-4-trifluoromethyl coumarin, as illustrated in example 6 below.

Thus, a competitive marker displaying for example strong affinity for the recognition sites of the second MIP can only be displaced by a particular target molecule having even stronger affinity for said recognition sites, whereas a competitive marker displaying lower affinity for the recognition sites of the second MIP will be displaced more easily by a target molecule, or even by a family of target molecules.

According to one embodiment of the invention, the competitive marker can be displaced by the target molecule(s) in the presence of very low contents of target molecule(s), and for example in the presence of contents below 1 μg/L or even below 100 ng/L.

Moreover, when the competitive marker used is detectable at very low contents, for example at contents below 500 ng/L, or even below 100 ng/L, and in particular when the competitive marker used is detectable at contents below the contents at which the target molecule(s) can be detected, detection stage (iv) according to the invention permits amplification of the signal for detection of the target molecule(s), and therefore improvement in the sensitivity of measurement.

Intrinsic Marker

According to a second embodiment, stage (iii) is based on manifestation of an interaction between the target molecule(s) and at least one intrinsic marker constituting the second MIP.

As stated previously, an intrinsic marker according to the invention can either constitute a part of the recognition sites of the target molecule(s) of the second MIP and thus participate in their recognition, or can be located near these sites.

In both of these cases, the intrinsic marker must, however, be able to interact with all or some of the target molecule(s) when the latter is/are extracted by the second MIP and must also be able to emit a detectable signal in consequence.

Detection of the target molecule(s) in stage (iii) is thus based, in this embodiment, on an interaction between at least one intrinsic marker and all or some of the target molecule(s).

According to one embodiment, this interaction can be noncovalent, preferably of the weak bond type, and for example of the hydrogen bond type.

It can notably be an interaction of the hydrogen bond type, which can for example result in a bathochromic shift (i.e. toward longer wavelengths) or hypsochromic shift (i.e. toward shorter wavelengths) of the wavelength detected.

Analysis

The method according to the invention can be intended for a qualitative analysis, i.e. solely intended to establish whether a target molecule or molecules is/are present in a particular medium.

The method according to the invention can also permit, apart from detection of the presence or absence of the target molecule(s), a semiquantitative, or even quantitative analysis of the latter, either by determining the quantity of competitive marker salted-out, or by analysis of the signal emitted by the intrinsic marker.

As stated previously, according to the variant of application employing a competitive marker, stage (iii) of detection in fact involves a competitive displacement of said marker by the target molecule(s), such that the number of moles of marker salted-out is a function of the number of moles of target molecule detected.

According to the variant employing an intrinsic marker, the variation of the signal is a function of the number of target molecules.

Thus, an analysis kit according to the invention can additionally comprise, according to one embodiment, a device for semiquantitative analysis and/or a device for quantitative analysis of the target molecule(s), based either on determination of the quantity of competitive marker salted-out, or on the analysis of the signal emitted by the intrinsic marker.

These devices can comprise, if necessary, calibrating means for correlating the quantity of competitive marker salted-out or the quantity of intrinsic marker emitting a detectable signal, with the quantity of target molecule(s).

According to one embodiment, it may be necessary to employ an additional stage of treatment, prior to the detection stage proper (iv). As an example of such treatments, we may notably mention washing, in particular in the case when the second polymerically imprinted polymer is packaged in the form of an SPE cartridge.

It is thus possible according to the invention for the concentration of target molecule(s) to be determined semiquantitatively, for example using, in stage (iv), a device for semiquantitative analysis comprising at least two analysis substrates containing second molecularly imprinted polymers of identical or different nature provided with competitive markers which may be identical or different or constituted of intrinsic markers which may be identical or different, detectable for example with the naked eye.

According to a variant of the invention, the markers can be identical, and the two analysis substrates can have different concentrations of markers corresponding to ranges of concentrations of target molecule(s) being determined.

According to an embodiment of the invention employing a competitive marker, notably illustrated in FIG. 2, the second molecularly imprinted polymer can be packaged in the form of a graduated SPE cartridge, and the competitive marker can be detectable with the naked eye, for example from its characteristic color. Detection stage (iv) is then made possible by the observation of disappearance of the color of the support, in the part of the support where the competitive displacement has already been manifested.

It is also possible according to the invention for the concentration of target molecule(s) to be determined semiquantitatively, for example by visualizing the progress of decoloration of the second MIP by a scale present on the support; said decoloration is a function of the concentration of target molecule.

Similarly, said semiquantitative analysis is also possible on the basis of coloration, using intrinsic markers.

Comparison of the signals detected following the salting-out of the competitive marker or the emission of the signal from the intrinsic marker, on each support, thus makes it possible to evaluate the content of target molecule(s) by locating it in ranges of concentration.

It is of course possible to envisage variable amounts of supports so as to provide coarser or finer ranges of contents.

For example, it is possible to provide from 1 to 20 separate supports, or even a virtually limitless number of supports, in the particular case when chips are used.

According to this variant of application, it is possible to vary the concentration of marker in the different supports by using second MIPs having variable densities of recognition sites, or by immobilizing a smaller quantity of imprint on the support, or by mixing MIPs with polymers identical to those constituting the matrix of the MIPs, but without imprints, at given ratios.

For application of the method of quantitative analysis, as the marker used according to the invention is by definition a detectable entity, it is also possible for the concentration of target molecule(s) to be determined accurately by measuring either the total quantity of competitive marker salted-out during detection stage (iii), or the signal emitted by the intrinsic marker in the course of detection stage (iii), and by referring, if necessary, to the result of a previous calibration.

All the methods of detection that can be used for this purpose are known by a person skilled in the art.

As pointed out previously, the methods of detection by visible colorimetry, by fluorescence and by visible UV are preferred within the scope of the present invention.

Applications

The analysis kit as claimed in the present invention can be used for numerous applications for purposes of analysis, and more particularly for the analysis of analytes present in complex media or present at trace levels in a sample.

Within the scope of the present invention, the analysis kit also means a diagnostic kit, a screening kit, a kit for analysis of pollutants, toxic products, medicinal products, contaminants, drugs, chemicals, perfumes and colorants.

It can notably be a diagnostic kit, a kit for analysis of pollutants, toxic products, medicinal products, contaminants, drugs, chemicals, perfumes, colorants, vitamins, proteins, amino acids and peptides, oligonucleotides, hormones, enzymes, biomarkers, metabolites, chemical or biochemical warfare agents, sugars, polysaccharides, neurotransmitters, mycotoxins, pesticides, fungicides, herbicides, insecticides, fertilizers, antibodies, molecules indicating the safety and/or quality of foodstuffs, steroids, drugs or reaction products or byproducts.

The target molecules that it is for example possible to analyze by means of the kit according to the invention are notably dopamine and its derivatives such as serotonin, L-tyrosine, 3,4-dihydroxy-phenylacetic acid or methoxytyramine, homocysteine, cysteine, glucose, cholesterol, testosterone, anabolic steroids, estradiol, citrosine, vitamin K, vitamin D, vitamin B12, atrazine, phenobarbital, chloramphenicol, propranolol, theophylline, diethylstilbene, progesterone, organophosphorus compounds, cocaine, THC, or ochratoxin A.

Thus, according to one embodiment of the invention, the analysis kit can be intended for the analysis of dopamine and/or its derivatives, as described above or for the analysis of ochratoxin A.

The invention will be better understood on reading the detailed description given below, nonlimiting examples of application, and on examining the appended drawings, which form an integral part of the description.

A kit for analysis of at least one target molecule comprising at least a first and a second molecularly imprinted polymers and at least one marker, can for example be offered to the user, in a box 1 containing, as can be seen in FIG. 4, two syringes 2 and 3 prepacked respectively with first and second MIPs 4 and 5, optionally a tube 6 (haemolysis tube or test tube) intended for receiving the elution solution from MIP 4, a container 7 containing the elution solution 8, a container 9 containing a washing solution 10, optional instructions for use 11 as well as optionally a container 12 containing reagents intended for adjusting the elution solution from MIP 4.

The kit can serve for a single use or on the contrary can contain sufficient amounts of reagents and of solution for elution to permit the analysis of several target molecules.

The syringes 2 and 3 can be made of glass or of plastic. They can have, as shown for syringe 2, a body 15 and a piston 14.

To use the kit shown in FIG. 4, the user introduces the solution to be analyzed 13 into the syringe 2 by means of a pipette, advantageously graduated, not shown in FIG. 4. Syringe 2 is shown in FIG. 4 in the presence of said solution to be analyzed 13. Once pressure has been applied by the user on piston 14 for evacuating the liquid containing the interfering substances, the target molecule is recovered by elution with solution 8, optionally after washing one or more times with solution 10.

The elution solution thus recovered is then either introduced directly into syringe 3 or recovered in tube 6 for adjustment of the medium using the reagent contained in container 12 and then introduced into syringe 3. The same piston 14 or another piston then serves for exerting the necessary pressure in syringe 3 to permit detection of the target molecule being sought, by emission of a signal after the elution solution recovered from the enrichment operation carried out using syringe 2 is brought in contact with the MIP 5.

According to the variant illustrated in FIG. 4, detection is visual, so that the second syringe is graduated and permits direct quantitative analysis, without passing through additional equipment, since all of the elution solution recovered from the enrichment operation carried out using syringe 2 has passed through the MIP 5 completely. According to this variant, the presence of the target molecule is reflected in the appearance, disappearance or change of color proportional to the quantity of molecule present.

In a variant that is not illustrated, the kit also comprises the detection system, when detection is not effected with the naked eye. Such a detection system can be a portable detector, for example a fluorescence detector or a UV detector. According to this variant, the syringe is advantageously graduated and can be carried in its entirety under a suitable lamp for visualizing the signal emitted.

Syringes 2 and 3 can be rinsed with a suitable solution, not shown in FIG. 4, in order to serve for further use.

A particular variant of use of the kit in FIG. 4 is shown in FIG. 5.

In this variant, the MIPs 4 and 5 are packaged in individual cartridges 17 and 18 with snap fitting respectively on syringe 16 and on cartridge 17.

Syringe 16 illustrates the first pretreatment stage and syringe 19, identical to or different from syringe 16, illustrates the detection stage where the solution filling syringe 19 is the solution eluted from the target molecule, favorable both for release of the target molecules from the MIP 4 and for interaction of the target molecule with the MIP 5.

FIG. 6 shows an example of an analysis kit 20 according to the invention in which the MIPs 21 and 22 are integrated in one and the same system, miniaturized or not, which can be a microfluidic system. A microfluidic system can more particularly be adapted to the treatment of small volumes of solution. Said microfluidic system can typically be in the form of an essentially flat system, with the format for example of a bank card.

In this system, the supports containing the MIPs 21 and 22 can be fixed in the system, or detachable. In this second case, the user can vary the pair of MIPs to be more particularly suited to the analysis in question.

Such a system can advantageously be transparent, and for example be made of glass or of plastic.

The containers containing the elution liquid and the washing liquid are not shown, and nor are the optional instructions for use.

The analysis kit 20 can for example have 3 inlets 23, 24 and 25 and 3 outlets 26, 27 and 28. Access to these inlets and/or outlets can be open or closed by valves 29, 30, 31, 32, 33 and 34.

To use the kit shown in FIG. 6, the user introduces, via inlet 24, the solution to be analyzed, after closing valves 29 and 31. This solution can for example be introduced by a pipette, by a syringe or by a micropump, not shown in FIG. 6.

The solution to be analyzed is thus brought in contact with the MIP 21 under pressure so that the liquid containing the interfering substances is evacuated via outlet 26 when valve 30 is open.

The user can carry out one or more washings of the MIP 21 by introducing, in the same way, a washing solution via inlet 24, with valves 29 and 31 closed and valve 30 open. The resultant solution or solutions, and if applicable the interfering substances, are thus directed to outlet 26.

The user can then continue with elution of the target molecule by introducing an eluting solution via inlet 24, with valves 29, 30 and 32 closed and valve 31 open, as well as one of valves 33 or 34. The elution solution thus recovered is then contacted directly with the MIP 22.

As a variant, the user can proceed to elution of the target molecule by introducing an eluting solution via inlet 24, with valves 29 and 31 closed, and valve 30 open. The elution solution is then recovered via outlet 26. It can then be introduced directly into the system via inlet 25, with valve 31 closed and valve 32 open as well as one of valves 33 or 34, or alternatively can be treated, before it is reintroduced into the system via inlet 25, with valve 31 closed and valve 32 open, as well as one of valves 33 or 34.

When the MIP 22 contains a competitive marker, the marker thus displaced in the preceding stage can be entrained with said solution to the outlet 27 or 28.

It is possible to add, at outlet 27 or 28, a detection system, not shown, when detection is not effected with the naked eye. Said system can be a portable detector, for example a fluorescence detector or UV detector.

When the MIP 22 is constituted of a marker, whether competitive or intrinsic, the user can directly detect the signal emitted by said marker (marker not displaced in the case of the competitive marker and marker emitting a signal in the case of the intrinsic marker). In this variant, said MIP can for example be provided with a graduated scale, not shown, for direct reading of the signal level emitted.

The system 20 can be rinsed with an appropriate solution, not shown in FIG. 6, in order to serve for further use. This solution can for example circulate between inlet 23 and outlet 27, with valves 29, 31 and 33 open and valves 30, 32 and 34 closed.

In the case when detection is not visual, the signal from the marker can be detected by positioning the system in its entirety in or under a suitable device for said detection, for example a fluorescent lamp. Even more particularly, when the system is flat, and is for example in the form of a card, it can be introduced via a slot into a suitable detection device.

Of course, other kits can also be used and other applications of the invention can be envisaged.

Throughout the text, including the claims, the expression “having one” must be understood as being synonymous with “having at least one”, unless specified otherwise.

### EXAMPLES

### Example 1

Synthesis of the monomer derived from N-tert-butoxycarbonyl-3,4-dihydroxyphenylethylamine. Synthesis of an MIP of dopamine starting from the monomer derived from N-tert-butoxycarbonyl-3,4-dihydroxyphenylethylamine designated “MIP No. 1” and from a matrix designated “Nonimprinted Material No. 1”, both materials being based on divinylbenzene (DVB). Synthesis of an MIP of dopamine starting from the monomer derived from N-tert-butoxycarbonyl-3,4-dihydroxyphenylethylamine designated “MIP No. 2” and from a matrix designated “Nonimprinted Material No. 2”, both materials being based on ethylene glycol dimethylacrylate (EGDMA).

### Monomer derived from N-tert-butoxycarbonyl-3,4-dihydroxyphenylethylamine

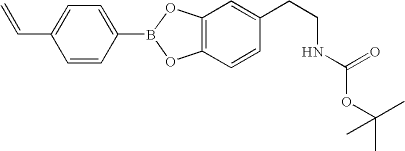

N-tert-butoxycarbonyl-3,4-dihydroxyphenylethylamine as described in Xu C., Xu K., Gu H., Zheng R., Liu H., Zhang X., Guo Z., Xu B. J. Am. Chem. Soc. 2004, 126, 9938 (0.609 g; 2.41 mmol) and 4-vinylphenyl boronic acid anhydride (0.312 g; 0.80 mmol) are dissolved in 60 mL of anhydrous toluene, then the mixture is refluxed for 3 hours under inert atmosphere. The particles that are insoluble when hot are filtered, then the toluene is evaporated. The oil obtained is diluted in the minimum amount of ethyl acetate, then cyclohexane (20 mL) is added to the mixture. We then obtain 0.812 g of a white powder (yield: 93%) corresponding to 5-[2-(N-tert-butoxycarbonyl)ethylamino]-2-(4-vinylphenyl)benzo[1,3,2]dioxoborole.

Matrixes Based on DVB

The divinylbenzene is washed several times with a basic solution saturated with NaCl to eliminate the inhibitor. It is dried over MgSO₄. The primer azobisisobutyronitrile (AIBN) is recrystallized from acetone.

The MIP No. 1 is prepared by mixing 725 mg of the monomer 5-[2-(N-tert-butoxycarbonyl)ethylamino]-2-(4-vinylphenyl)benzo[1,3,2]dioxoborole, 6.2 g of 80% divinylbenzene, 1.1 g of 2-carboxyethyl acrylate in 7.2 mL of anhydrous chloroform. The mixture is degassed by bubbling with nitrogen for 10 minutes, then 89 mg of AIBN is added. Polymerization is carried out at 50° C. for 48 hours to form a monolithic white substance.

The unimprinted material No. 1 is prepared by mixing 1.98 g of styrene, 6.2 g of 80% divinylbenzene, 1.1 g of 2-carboxyethyl acrylate in 7.2 mL of anhydrous chloroform. The mixture is degassed by bubbling with nitrogen for 10 minutes, then 89 mg of AIBN is added. Polymerization is carried out at 50° C. for 48 hours to form a monolithic white substance.

Matrixes Based on EGDMA

The ethylene glycol dimethylacrylate is washed several times with a basic solution saturated with NaCl to eliminate the inhibitor. It is dried over MgSO₄. The primer azobisisobutyronitrile (AIBN) is recrystallized from acetone.

The MIP No. 2 is prepared by mixing 626 mg of the monomer 5-[2-(N-tert-butoxycarbonyl)ethylamino]-2-(4-vinylphenyl)benzo[1,3,2]dioxoborole, 6.48 g of ethylene glycol dimethylacrylate, 940 mg of 2-carboxyethyl acrylate in 9 mL of anhydrous chloroform. The mixture is degassed by bubbling with nitrogen for 10 minutes, then 77 mg of AIBN is added. Polymerization is carried out at 50° C. for 48 hours to form a monolithic white substance.

The unimprinted material No. 2 is prepared by mixing 1.98 g of styrene, 7.55 g of ethylene glycol dimethylacrylate, 1.1 g of 2-carboxyethyl acrylate in 7.2 mL of anhydrous chloroform. The mixture is degassed by bubbling with nitrogen for 10 minutes, then 89 mg of AIBN is added. Polymerization is carried out at 50° C. for 48 hours to form a monolithic white substance.

The matrixes prepared above are submitted to coarse grinding and suspended in 80 mL of a 9% methanol solution in hydrochloric acid for 24 hours. Once filtered, the powder is resuspended in 80 mL of methanol for 24 hours. The filtered powder is then ground and sieved. Particles with size between 25 and 45 μm are introduced into an HPLC column 150×4.6 mm, then compacted by pressing and washed with a mixture of 5% acetic acid in acetonitrile/H₂O (97.5/2.5), then with methanol to investigate the recognition of dopamine in HPLC. We thus have 4 HPLC columns. SPE cartridges with particles 25-45 μm with each matrix are prepared in parallel.

### Example 2

### Recognition Properties with Respect to Dopamine by HPLC

Recognition takes place according to the following scheme.

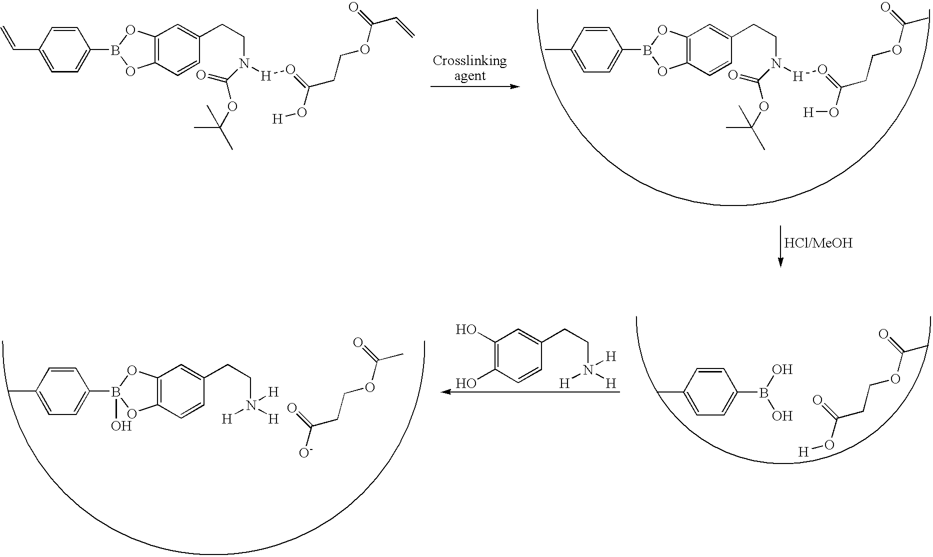

A 1 mM solution of dopamine hydrochloride in methanol is injected in the four columns filled respectively with imprint No. 1, unimprinted material No. 1, imprint No. 2 and unimprinted material No. 2.

The eluent used is a 95/5 mixture of MeOH/acetate buffer 10 mM at pH=5 with a flow rate of 1 mL/min. Detection of dopamine is performed at 281 nm. The injection volumes are 20 μL and 5 μL of a solution of acetone in methanol. Acetone is used for determining the dead volume of the column.

The values of k′ (capacity factor) and of IF (imprinting factor) are determined for evaluating the recognition of dopamine on all the matrixes.

Matrixes Based on DVB

| Analyte | Dopamine 0.82 mM |
|---|---|
| ${k}_{MIP No . 1}^{′} = \frac{{t}_{analyte} - {t}_{acetone}}{{t}_{acetone}}$ | 11.06 |
| ${k}_{nonimprinted material No . 1}^{′} = \frac{{t}_{analyte} - {t}_{acetone}}{{t}_{acetone}}$ | 4.63 |
| $IF = \frac{{k}_{MIP No . 1}^{′}}{{k}_{nonimprinted material No . 1}^{′}}$ | 2.39 |

Matrixes Based on EGDMA

| Analyte | Dopamine 0.91 mM |
|---|---|
| ${k}_{MIP No . 2}^{′} = \frac{{t}_{analyte} - {t}_{acetone}}{{t}_{acetone}}$ | 29.83 |
| ${k}_{nonimprinted material No . 2}^{′} = \frac{{t}_{analyte} - {t}_{acetone}}{{t}_{acetone}}$ | 12.48 |
| $IF = \frac{{k}_{MIP No . 2}^{′}}{{k}_{nonimprinted material No . 2}^{′}}$ | 2.39 |

We observe that the dopamine is recognized by both MIPs. However, the dopamine is retained on MIP No. 2 more than on MIP No. 1 (higher k′).

### Example 3

### Selectivity of the Various Matrixes with Respect to Several Compounds by HPLC

Various 1 mM solutions of several compounds in methanol are injected in the four columns filled respectively with MIP No. 1, unimprinted material No. 1, MIP No. 2 and unimprinted material No. 2.

The eluent used is a 95/5 mixture of MeOH/acetate buffer 10 mM at pH=5 with a flow rate of 1 mL/min. Detection of the compounds is effected at 281 nm. The injection volumes are 20 μL.

The values of k′ (capacity factor) and of IF (imprinting factor) are determined for evaluating the recognition of the compounds on all the matrixes.

Matrixes Based on DVB

| Analyte | k′_{MIP No. 1} | k′_{unimprinted material No. 1} | IF |
|---|---|---|---|
| Dopamine 0.82 mM | 11.06 | 4.63 | 2.39 |
| Catechol 0.9 mM | 2.20 | 0.43 | 1.25 |
| 3,4-Dihydroxyphenylacetic | 0 | 0 | — |
| acid 0.97 mM | | | |
| L-Tyrosine 0.94 mM | 0.151 | 0.136 | 1.1 |
| Serotonin 0.80 mM | 6.315 | 6.44 | 0.98 |

Matrixes Based on EGDMA

| Analyte | k′_{MIP No. 2} | k′_{unimprinted material No. 2} | IF |
|---|---|---|---|
| Dopamine 0.82 mM | 29.83 | 12.48 | 2.39 |
| 6,7-Hydroxy-4-trifluoro- | 24.61 | 1.85 | 13.3 |
| methyl coumarin 0.97 mM | | | |
| Catechol 1.21 mM | 0.76 | 1.28 | 0.59 |
| 3,4-Dihydroxyphenylacetic | 0 | 0 | — |
| acid 0.71 mM | | | |
| L-Tyrosine 1.16 mM | 0.18 | 3.02 | 0.06 |
| Serotonin 1.07 mM | 15.70 | 13.21 | 1.19 |

It is found by HPLC that the molecules containing an acid function are not retained at all on the imprints, in contrast to dopamine.

The imprints are selective for dopamine relative to molecules of similar structure. Imprint No. 1 is more selective than imprint No. 2 based on the values of the imprinting factor of serotonin on the two imprints.

6,7-Hydroxy-4-trifluoromethyl coumarin is retained less well on imprint No. 2 than dopamine, but some retention is observed on this imprint. This molecule can be used as marker for investigations of displacement.

### Example 4

### Recognition of Dopamine by SPE

Two SPE cartridges are made by introducing 200 mg of each of the imprinted matrixes (imprints No. 1 and No. 2) between two frits. Extraction of dopamine is then performed. For this, 5 mL of a solution of 10 mM phosphate buffer at pH 7 are percolated through these cartridges followed by 5 mL of MilliQ water and 5 mL of MeOH for conditioning the cartridges before introducing the dopamine. Then 500 μL of MeOH-acetate buffer solution pH=5 10 mM (80/20, v/v) doped at 10 μg/mL of dopamine are percolated through the SPE cartridges. Several 3 mL fractions of MeOH-acetate buffer solution pH=5 (80/20, v/v) are used as washing solution. Then 2×2 mL of MeOH containing 0.025% of acetic acid are percolated. The various fractions are then analyzed by HPLC-UV.

The table gives the recovery rates (%) of dopamine obtained during this extraction.

Dopamine recovery rate, %

| Fraction | Imprint No. 1 | Imprint No. 2 |
|---|---|---|
| Percolation | 0 | 0 |
| Washing 1 | 0 | 0 |
| Washing 2 | 0 | 0 |
| Washing 3 | 69 | 0 |
| Washing 4 | 0 | 0 |
| Washing 5 | 0 | 0 |
| 2 mL MeOH-AA 0.025% | 0 | 49 |
| 2 mL MeOH-AA 0.025% | 0 | 0 |

In these conditions of use, a difference is observed in the behavior of the two matrixes with respect to the retention of dopamine.

### Example 5

### Selectivity of the Imprint by SPE

The selectivity of the imprinted matrix was tested by adding a molecule similar to dopamine during the percolation phase. 3,4-Dihydroxy-phenylacetic acid was selected as the test molecule. After conditioning the cartridges, 500 μL of MeOH containing 5 μg of the acid and 5 μg of dopamine are introduced. Several washing fractions (2 mL of MeOH-acetate buffer pH=5 10 mM (95/5, v/v)) are then used, followed by elution with MeOH-0.05% acetic acid.

The table shows the recovery rate (%) of dopamine and of 3,4-dihydroxyphenylacetic acid obtained during this extraction.

Recovery rate, %

| Fraction | Dopamine | 3,4-Dihydroxyphenylacetic acid |
|---|---|---|
| Percolation | 0 | 6 |
| Washing 1 | 0 | 82 |
| Washing 2 | 0 | 0 |
| Washing 3 | 0 | 0 |
| Washing 4 | 0 | 0 |
| Washing 5 | 0 | 0 |
| Elution | 100 | 0 |

3,4-Dihydroxyphenylacetic acid is not retained on the imprinted matrix and leaves once the products are introduced.

As in HPLC, it is found that the molecule containing an acid function is not retained on the imprints at all, in contrast to dopamine. The imprints are selective for dopamine relative to molecules of similar structure.

### Example 6

### Displacement of 6,7-hydroxy-4-trifluoromethyl coumarin by SPE

Test A:

Two SPE cartridges containing 70 mg of imprinted matrix are prepared. After conditioning, the cartridges are saturated with 500 μg of 6,7-hydroxy-4-trifluoromethyl coumarin in methanol. On the cartridge designated “developer”, 4 fractions of 1 mL containing 100 μg of dopamine and 10 μg of 6,7-hydroxy-4-trifluoromethyl coumarin are introduced. On the second cartridge designated “control”, only 10 μg of 6,7-hydroxy-4-trifluoromethyl coumarin is introduced. 1 mL fractions containing 10 μg of 6,7-hydroxy-4-trifluoromethyl coumarin in MeOH are then percolated through the two cartridges and each fraction is analyzed. The following table gives the amounts of 6,7-hydroxy-4-trifluoromethyl coumarin obtained in these various fractions.

| | Recovery rate, % of 6,7-hydroxy-4- | |
|---|---|---|
| | trifluoromethyl coumarin | |
| Fraction | Developer | Control |
| Last introduction in dopamine | 53 | 27 |
| Fraction 1 | 49 | 23 |
| Fraction 2 | 43 | 20 |
| Fraction 3 | 44 | 19 |
| Fraction 4 | 27 | 18 |

We observe that the introduction of dopamine leads to a higher recovery rate of 6,7-hydroxy-4-trifluoromethyl coumarin on the developer than on the control.
Test B:

Two SPE cartridges containing 25 mg of imprinted matrix are prepared. After conditioning, 35 μg of 6,7-hydroxy-4-trifluoromethyl coumarin contained in 1 mL of methanol is percolated followed by 1 mL of MeOH. On the cartridge designated “developer”, 1 mL of MeOH containing either 100 μg or 50 μg of dopamine is introduced. On the second cartridge designated “control”, only 1 mL of MeOH is introduced. 1 mL fractions of MeOH are then percolated through the two cartridges. The graph in FIG. 3 shows the amounts of 6,7-hydroxy-4-trifluoromethyl coumarin obtained in these various fractions during introduction of 100 μg of dopamine. The following table shows the amounts of 6,7-hydroxy-4-trifluoromethyl coumarin obtained in these various fractions after percolation of 50 μg of dopamine.

| | Recovery rate, % of 6,7-hydroxy-4- | |
|---|---|---|
| | trifluoromethyl coumarin | |
| Fraction | Developer | Control |
| Introduction of 50 μg of dopamine | 5 | 5 |
| Fraction 1 | 6 | 4 |
| Fraction 2 | 6 | 3 |
| Fraction 3 | 4 | 2 |
| Fraction 4 | 3 | 2 |

After introduction of the dopamine on the developer, we observe a change in the amount of 6,7-hydroxy-4-trifluoromethyl coumarin due to displacement of the latter by the dopamine for the two concentrations tested, as well as decoloration of the cartridge as a function of the amount of dopamine.

### Example 7

### Synthesis of an MIP of Ochratoxin A “MIP No. 3” Based on Ethylene Glycol Dimethylacrylate (EGDMA)

MIP No. 3

MIP No. 3 is prepared by mixing 505 mg of ochratoxin A, 4.95 g of ethylene glycol dimethylacrylate, 430 mg of methacrylic acid in 6.9 mL of anhydrous acetonitrile. The mixture is degassed by bubbling with nitrogen for 10 minutes, then 56 mg of AIBN is added. Polymerization is carried out at 50° C. for 48 hours to form a monolithic white substance.

MIP No. 3 prepared above is ground and then sieved. Particles with size between 25 and 45 μm are introduced in an HPLC column 150×4.6 mm then compacted by pressing and washed with a mixture 5% acetic acid in acetonitrile/H₂O (97.5/2.5) and then acetonitrile. The particles of 25-45 μm are introduced in SPE cartridges.

### Example 8

Synthesis of the monomer (E)-1-(2-methacryloxyethyl)-3-[4-(4-nitrophenyldiazenylphenyl]thiourea. Synthesis of an MIP of ochratoxin A (OTA) starting from (E)-1-(2-methacryloxyethyl)-3-[4-(4-nitrophenyldiazenylphenyl]thiourea “MIP No. 4” based on ethylene glycol dimethylacrylate (EGDMA).

### Monomer derived from Disperse Orange 3 ((E)-1-(2-methacryloxyethyl)-3-[4-(4-nitrophenyldiazenylphenyl]thiourea)

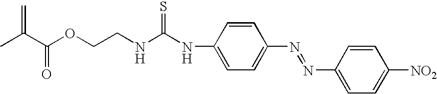

First 2.21 g of Disperse Orange (95%) and then 22 mL of dry THF are introduced in a two-necked flask under nitrogen. Add 600 μL of phenyl chlorothionocarbonate. Stir at room temperature for 2 hours. Using TLC (eluent: DCM), the starting product is no longer observed. Filter on Celite. A clear red solution is obtained. Evaporate, then add, under nitrogen, 24 mL of toluene, 605 μL of triethylamine and 439 μL of HSiCl₃. Stir at room temperature for 4 h 45 min then filter on Celite.

Evaporate the filtrate. After purification on a silica column, the expected product thioisocyanate of Disperse Orange 3 is obtained at a yield of 37% (460 mg). Dissolve the latter in 40 mL of DCM. Add 323 μL of triethylamine (1.1 eq) and, under nitrogen, add 2-aminoethylmethacrylate hydrochloride (90%, 428 mg, 1.1 eq.). Stir at room temperature overnight. Evaporate and purify on a silica column, using as eluent dichloromethane (DCM) then an 80/20 mixture of DCM/AcOEt. We obtain 326 mg of monomer of Disperse Orange 3, i.e. 37% yield.

MIP No. 4

MIP No. 4 is prepared by mixing 125 mg of ochratoxin A, 1.23 g of ethylene glycol dimethylacrylate, and 128 mg of (E)-1-(2-methacryloxyethyl)-3-[4-(4-nitrophenyldiazenylphenyl]thiourea in 1.7 mL of anhydrous acetonitrile. The mixture is degassed by bubbling with nitrogen for 10 minutes, then 14 mg of AIBN is added. Polymerization is carried out at 50° C. for 48 hours to form a monolithic orange substance.

The MIP No. 4 prepared above is ground and then sieved. The particles with size between 25 and 45 μm are introduced in an HPLC column 150×4.6 mm then compacted by pressing and washed with a mixture of 5% acetic acid in acetonitrile/H₂O (97.5/2.5), then with acetonitrile. The particles of 25-45 μm are introduced in SPE cartridges.

### Example 9

### Recognition of OTA by SPE

Two SPE cartridges are made by introducing 50 and 100 mg of each of the imprinted matrixes (imprint No. 3 and No. 4) between two frits. Extraction of OTA is then performed.

Protocol A

5 mL of white wine doped with OTA is percolated through the SPE cartridge of 50 mg of imprint No. 3. After washing several times, an elution fraction containing OTA at 99% is obtained at the end of the protocol.

The fraction containing OTA is percolated through the SPE cartridge of 100 mg of imprint No. 4. A brown coloration is then observed, proportional to the amount of OTA present in the elution fraction.

Protocol B

5 mL of white wine doped with OTA is percolated through the SPE cartridge of 100 mg of imprint No. 4. No coloration is observed: the orange coloration characteristic of Disperse Orange 3 is unchanged.

It therefore appears that the pretreatment of the sample is essential.

1. A kit for analysis of at least one target molecule, comprising
at least a first and a second molecularly imprinted polymers which are chemically identical or different, and are capable of interacting with the target molecule(s), and
at least one marker, wherein said marker is a competitive marker or is capable (i) of interacting with at least one of recognition sites of the target molecule(s) of the second molecularly imprinted polymer and (ii) of being displaced from said recognition site(s) by the target molecule(s) on bringing said second molecularly imprinted polymer in contact with said target molecule(s), or wherein said marker is either an intrinsic marker or a constitutive unit of the second molecularly imprinted polymer, and is capable (i′) of interacting with at least one of the target molecule(s) when said target molecule(s) interacts/interact with at least one of the recognition sites of said target molecule(s) and (ii′) of emitting, in consequence, a detectable signal.

2. The kit according to claim 1, wherein the competitive marker and the second molecularly imprinted polymer are packaged separately.

3. The kit according to claim 1, wherein the competitive marker and the second molecularly imprinted polymer are packaged together wherein that the second molecularly imprinted polymer is in at least one of the recognition sites of the target molecule(s), with said marker.

4. The analysis kit according to claim 1, wherein the second molecularly imprinted polymer is of different chemical nature than the first molecularly imprinted polymer.

5. A kit for analysis of at least one target molecule comprising at least a first and a second molecularly imprinted polymers that are chemically different, capable of interacting with the target molecule(s) and at least one marker, said marker being capable of emitting a detectable signal on bringing said second molecularly imprinted polymer in contact with said target molecule(s).

6. The analysis kit according to claim 1, wherein the first molecularly imprinted polymer extracts the target molecule(s), by molecular recognition of the target molecule(s).

7. The analysis kit according to claim 1, wherein the second molecularly imprinted polymer detects the target molecule(s).

8. The analysis kit according to claim 1, wherein the marker, the signal or a variation of signal emitted by the marker is detected by visible colorimetry, by radiochemistry, by nuclear medicine, by imaging, by resonance (MRI), by X-rays, by diffusion of light, by mass spectrometry, by spectroscopy, by infrared spectroscopy, by surface plasmon resonance spectroscopy, by chemiluminescence, by interference spectroscopy and refraction spectroscopy, by Raman scattering, by ultrasound, by radioactivity, by refractometry, by optical, piezoelectric, magnetic, or acoustic detection, by electrochemistry, by conductivity, by pH measurement, by biological means, or by the naked eye.

9. The analysis kit according to claim 1, wherein the kit is for the analysis of a family of target molecules.

10. The analysis kit according to claim 1, wherein the kit is analyzes dopamine, and derivatives thereof selected from the group consisting of serotonin, L-tyrosine, 3,4-dihydroxyphenylacetic acid and methoxytyramine, homocysteine, cysteine, glucose, cholesterol, testosterone, anabolic steroids, estradiol, citrosine, vitamin K, vitamin D, vitamin B12, atrazine, phenobarbital, chloramphenicol, propranolol, theophylline, diethylstilbene, progesterone, organophosphorus compounds, cocaine, THC, and ochratoxin A.

11. The analysis kit according to claim 1, wherein the first and/or second molecularly imprinted polymers employ, during their polymerization stage, at least 5-[2-(N-tert-butoxycarbonyl)ethylamino]-2-[4-vinylphenyl]benzo[1,3,2]dioxoborole.

12-13. (canceled)

14. The analysis kit according to claim 1, wherein the kit additionally comprises a device for semiquantitative analysis and/or a device for quantitative analysis of the target molecule(s), by determination of the quantity of competitive marker salted-out, or by analysis of the signal emitted by the intrinsic marker.

15. The analysis kit according to claim 14, wherein the device for semiquantitative analysis comprises at least two analysis substrates comprising second polymerically imprinted polymers, of identical or different nature, comprising detectable competitive markers, which are identical or different, or comprising detectable intrinsic markers which are identical or different.

16. (canceled)

17. The analysis kit according to claim 1, wherein the kit is a diagnostic kit, a kit for analysis of pollutants, toxic products, medicinal products, contaminants, drugs, chemicals, perfumes, colorants, vitamins, proteins, amino acids and peptides, oligonucleotides, hormones, enzymes, biomarkers, metabolites, chemical or biochemical warfare agents, sugars, polysaccharides, neurotransmitters, mycotoxins, pesticides, fungicides, herbicides, insecticides, fertilizers, antibodies, molecules indicating the safety and/or quality of foodstuffs, steroids, drugs or reaction products or byproducts.

18. A method of analysis of at least one target molecule comprising a kit according to claim 1.

19. The method according to claim 18, wherein the target molecule(s) is/are present in a solution.

20. A method of analyzing at least one target molecule that is present in a solution, comprising at least:
(i) contacting said solution with a first molecularly imprinted polymer that interacts with the target molecule(s) in conditions suitable for removal of the target molecule(s),
(ii) forming a purified solution, optionally enriched in target molecule(s), starting from the target molecule(s) isolated in (i),
(iii) contacting said purified solution, optionally enriched in target molecule(s), with a second molecularly imprinted polymer, interacting with said target molecule(s), and contacting said purified solution, in at least one of recognition sites of the target molecule(s), with at least one competitive marker or marker that can be displaced from said recognition site(s) by the target molecule(s) in carrying out said (iii), or with at least one intrinsic marker or marker that is able to interact with all or some of said target molecule(s) when said target molecule(s) interacts/interact with at least one of the recognition sites of said target molecule(s) and emit in consequence a detectable signal and
(iv) carrying out qualitative, quantitative and/or semiquantitative detection of the target molecule(s), through the detection either of the competitive marker thus displaced, or of the signal thus emitted by the intrinsic marker.

21. The method according to claim 20, wherein (ii) is carried out by bringing the target molecule(s) isolated in stage (i) in contact with a medium that promotes rupture of their interaction with the first molecularly imprinted polymer.

22. The method according to claim 20, wherein said purified solution, optionally enriched in target molecule(s), is directly contacted with said second molecularly imprinted polymer.

23. The method according to claim 20, wherein said purified solution, optionally enriched in target molecule(s), is treated before it is brought in contact with said second molecularly imprinted polymer.

24. (canceled)
